(19) United States
(12) Patent Application Publication
(10) Pub. No.: US 2024/0395393 A1
(43) Pub. Date: Nov. 28, 2024

(54) METHOD FOR INPUTTING MEDICAL-IMAGE DIAGNOSTIC REPORT USING THREE-DIMENSIONAL TEXT DATA STRUCTURE AND COMPUTER-READABLE RECORDING MEDIUM ON WHICH PROGRAM FOR PERFORMING SAME IS RECORDED

(71) Applicant: Hoon Sik JUNG, Yangsan-si (KR)

(72) Inventor: Hoon Sik JUNG, Yangsan-si (KR)

(21) Appl. No.: 18/675,316

(22) Filed: May 28, 2024

Related U.S. Application Data
(63) Continuation of application No. PCT/KR2024/004823, filed on Apr. 11, 2024.

(30) Foreign Application Priority Data
May 25, 2023 (KR) 10-2023-0067803

(51) Int. Cl. G16H 30/40

(52) U.S. Cl. CPC G16H 30/40

(57) ABSTRACT

A method for inputting a medical-image diagnostic report using a three-dimensional text data structure, includes: generating, by at least one processor, text information for medical-image reporting in a three-dimensional data structure; providing, by the at least one processor, a user interface for the text information; and inputting, by the at least one processor, only selection information selected from a user terminal within the text information provided to the user interface into a diagnostic report input field.

### CROSS-REFERENCE TO PRIOR APPLICATIONS

This application is a Continuation Application of PCT Internation Application No. PCT/KR2024/004823 (filed on Apr. 11, 2024), which claims priority to Korean Patent Application No. 10-2023-0067803 (filed on May 25, 2023), which are all hereby incorporated by reference in their entirety.

### BACKGROUND

The present disclosure relates to a method for inputting a medical-image diagnostic report using a three-dimensional text data structure that allows a medical staff to efficiently input his or her findings and diagnosis, and a computer-readable recording medium on which a program for performing the same is recorded.

A radiology examination is a process of diagnosing an examinee's disease or evaluating a treatment process by visualizing an inside of the examinee's body using various medical imaging devices such as X-rays, CT, MRI, and ultrasound. The radiology examination includes examination request, examination progress, medical image generation and storage, medical image analysis, findings, and diagnosis processes.

First, a clinician makes a request for an appropriate radiology examination according to the examinee's symptoms, treatment process, and expected disease. Then, a radiologic technologist collects the examinee's medical information and the clinician's request in an examination room to carry out an appropriate examination. Medical images generated upon the completion of the relevant examination are stored on a server of a picture archiving and communicating system (PACS).

Then, a radiologist may access medical images in a reading room through the picture archiving and communicating system (PACS) to analyze the medical images. The radiologist may use his or her medical knowledge and experience to determine the presence, location, size, and form of a disease in the medical images, diagnose the examinee's disease, or evaluate the treatment process. Furthermore, based on a result of the analysis, the radiologist writes a diagnostic report including image findings, diagnosis details, and necessary recommended actions. The written diagnostic report is stored in the picture archiving and communicating system (PACS), and stored and shared in an electronic medical record (EMR) system linked that is linked with it.

Then, the clinician who has previously made a request for a radiology examination can access the diagnostic report through the picture archiving and communicating system (PACS) and the electronic medical record (EMR) system, and based thereon, carry out a face-to-face consultation with the examinee, and determine additional examinations and treatment directions.

Recently, various factors such as advances in medical imaging technology, population aging, the development of health insurance systems leading to the convenient and affordable realization of radiological examinations, increased awareness of public health, and government-led screening services, including radiology examinations such as cancer screenings, contribute to the increasing trend of radiological examinations. However, because the release of radiologists who read medical images in the radiology department is constant every year, a number of examinations to be processed per each radiologist is increasing. Furthermore, because a number of medical images per each examination has increased significantly, the work of the radiologist is increasing, which may reduce the accuracy of reading in the future.

In order to solve the foregoing problems, measures such as introduction of artificial intelligence technology, introduction of a remote interpretation system that requests reading from an external medical imaging specialist, development of standardized guidelines, education, training, and collaboration of radiologists have been proposed. However, although the above-mentioned measures are helpful in coping with the increasing number of radiology examinations, practical and groundbreaking technology for a radiologist to efficiently input a diagnostic report is still required in this technical field.

Diagnostic report input methods used in the related art include a keyboard method, a template method, a medical transcription method, a voice recognition method, and the like. The keyboard method is the most traditional method, but it takes a long time to input a diagnostic report, typos may occur, and as the number of readings increases, it may cause fatigue for the radiologist. The template method has a one-dimensional data structure, so it is difficult to find the corresponding template as the number of data increases, and it is difficult for the radiologist to respond to various findings or diagnoses because he or she must select from limited options within the template. Furthermore, there is an inconvenience in that when the radiologist moves to a medical institution or the picture archiving and communicating system (PACS) is changed or updated, the template must be reconfigured appropriately.

In addition, the medical transcription method, which is a method mainly used in university hospitals, requires hiring a professional capable of transcription, and is inconvenient as the radiologist has to recheck the input, and therefore is highly likely to be replaced by voice recognition in the future. The voice recognition method may reduce a diagnostic report input time by converting voice into real-time text through a program, but it may be interrupted by noise other than the radiologist's voice, and may tend to rely on the radiologist's instantaneous judgment, so unnecessary information may be input or essential information may not be input into the diagnostic report.

### SUMMARY

The present disclosure aims to solve the foregoing problems, and in order to overcome the limitations of the existing diagnostic report input method and allow a radiologist to enter a diagnostic report efficiently, an aspect of the present disclosure is to provide a method for inputting a medical-image diagnostic report using a three-dimensional text data structure in which text information for medical-image reporting is generated in a three-dimensional data structure, and only selection information selected from a user terminal within text information provided to a user interface is input into a diagnostic report input field, and a computer-readable recording medium on which a program to perform this is recorded.

Technical problems to be solved in the present disclosure are not limited to the above-mentioned problems and other technical problems which are not mentioned herein will definitely be understood by those skilled in the art from the following description.

In order to achieve the foregoing objectives, a method for inputting a medical-image diagnostic report using a three-dimensional text data structure according to the present disclosure provides a text information generation step of generating, by at least one processor, text information for medical-image reporting in a three-dimensional data structure; a user interface provision step of providing, by the at least one processor, a user interface for the text information; and a selection information input step of inputting, by the at least one processor, only selection information selected from a user terminal within the text information provided to the user interface into a diagnostic report input field.

In order to achieve the foregoing objectives, the present disclosure relates to a computer-readable recording medium on which a program for performing the medical-image diagnostic report input method using a three-dimensional text data structure is recorded.

As described above, according to the present disclosure, text information for medical-image reporting may be generated in a three-dimensional data structure, and only selection information selected from a user terminal within text information provided to a user interface may be input into a diagnostic report input field, thereby having an effect of overcoming the limitations of the existing diagnostic report input method for medical images and allowing a radiologist to input diagnostic findings efficiently.

In addition, the present disclosure may allow compatibility with other programs such as a picture archiving and communicating system (PACS) and an electronic medical record (EMR) system, thereby replacing or combining conventional keyboard input, voice recognition, and template-based input methods therewith to allow a radiologist to quickly and accurately input a diagnostic report.

In addition, the present disclosure may be used as a writing guide when a radiologist with relatively limited clinical experience writes diagnostic findings subsequent to medical-image review, thereby having an effect of improving the quality of a diagnostic report regardless of clinical experience.

The effects of the present disclosure are not limited to the above-mentioned effects, and other effects that are not mentioned herein will be clearly understood by those skilled in the art from the following detailed description and appended claims.

### BRIEF DESCRIPTION OF THE DRAWINGS

FIG. 1 is a flowchart showing a method for inputting a medical-image diagnostic report using a three-dimensional text data structure of the present disclosure.

FIG. 2 is a diagram showing a three-dimensional text data structure according to an embodiment of the present disclosure.

FIG. 3 is a diagram showing text information and a text information input field according to an embodiment of the present disclosure.

FIG. 4 is a diagram showing medical images for a chest examination according to an embodiment of the present disclosure.

FIG. 5 is a diagram showing a primary layer of one set according to an embodiment of the present disclosure.

FIG. 6 is a diagram showing a secondary layer of one set according to an embodiment of the present disclosure.

FIG. 7 is a diagram showing a primary layer of a second set according to an embodiment of the present disclosure.

FIG. 8 is a diagram showing a picture archiving and communicating system (PACS) into which selection information is input according to an embodiment of the present disclosure.

FIG. 9 is a diagram showing an electronic medical record (EMR) system into which selection information is input according to an embodiment of the present disclosure.

FIG. 10 is a diagram showing a Windows notepad into which selection information is input according to an embodiment of the present disclosure.

FIG. 11 is a diagram showing an Excel document into which selection information is input according to an embodiment of the present disclosure.

### DETAILED DESCRIPTION

Although the terms used herein are selected from generally known and used terms considering their functions in this disclosure, they may be modified depending on the intentions of persons skilled in the art, practices, precedents, or the advent of new technology. Furthermore, in specific cases, some terms may be arbitrarily chosen by the applicant, and the meanings of these terms are detailed in the corresponding sections of this disclosure. Accordingly, the terms used herein should be understood not simply by the actual terms used but by the meaning lying within and the description disclosed herein.

Unless defined otherwise, the terms used herein including technological or scientific terms have the same meaning that is generally understood by those skilled in the art to which the present disclosure pertains. The terms used herein shall not be interpreted not only based on the definition of any dictionary but also the meaning that is used phase the field to which the invention pertains, and shall not be interpreted too ideally or formally unless clearly defined herein.

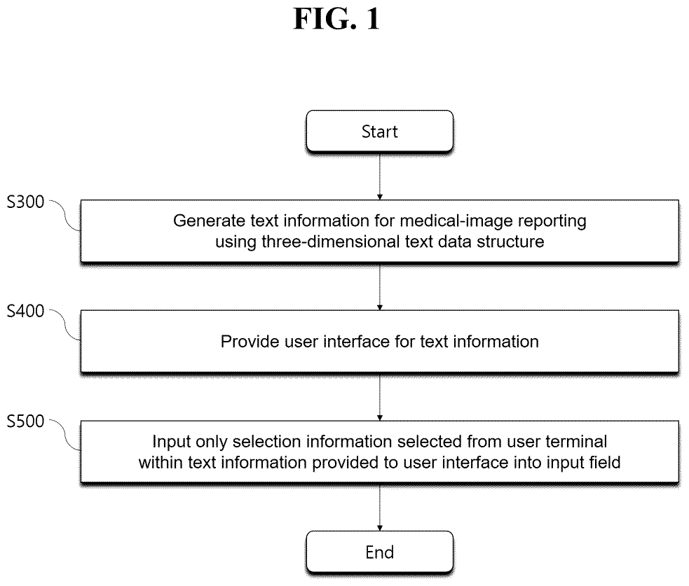

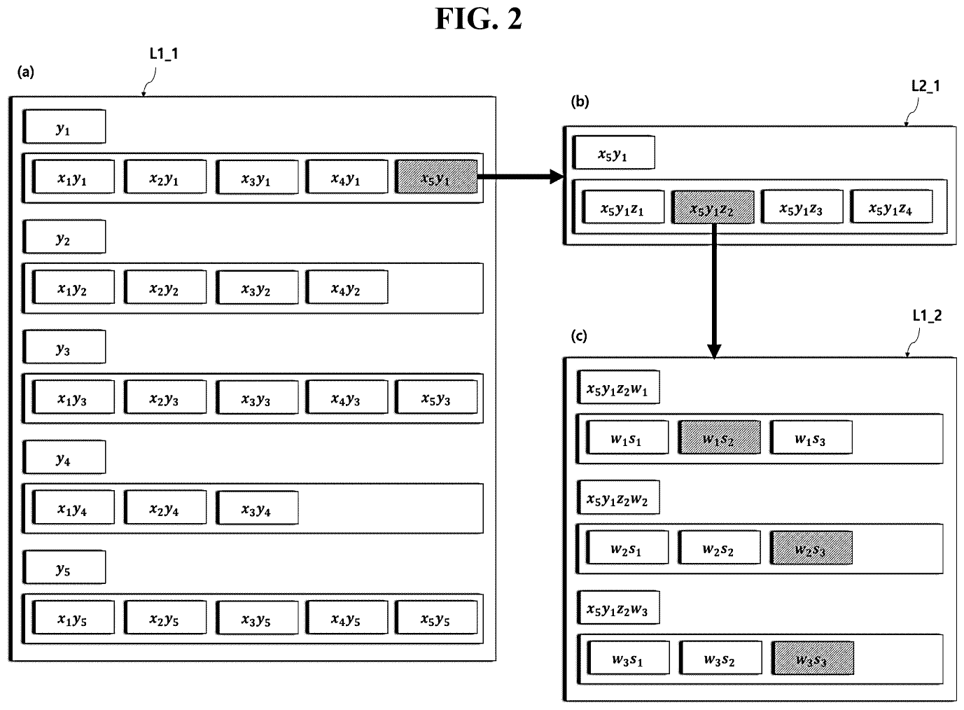

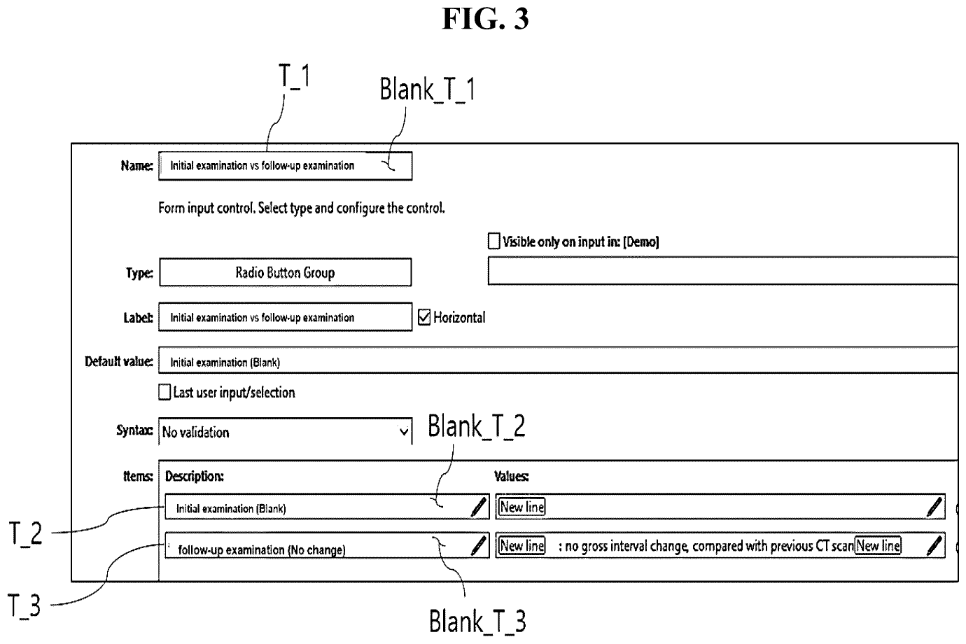

Hereinafter, an embodiment according to the present disclosure will be described in detail with reference to the accompanying drawings. FIG. 1 is a flowchart showing a method for inputting a medical-image report using a three-dimensional text data structure of the present disclosure. FIG. 2 is a diagram showing a three-dimensional text data structure according to an embodiment of the present disclosure. FIG. 3 is a diagram showing text information T and a text information input field Blank_T according to an embodiment of the present disclosure.

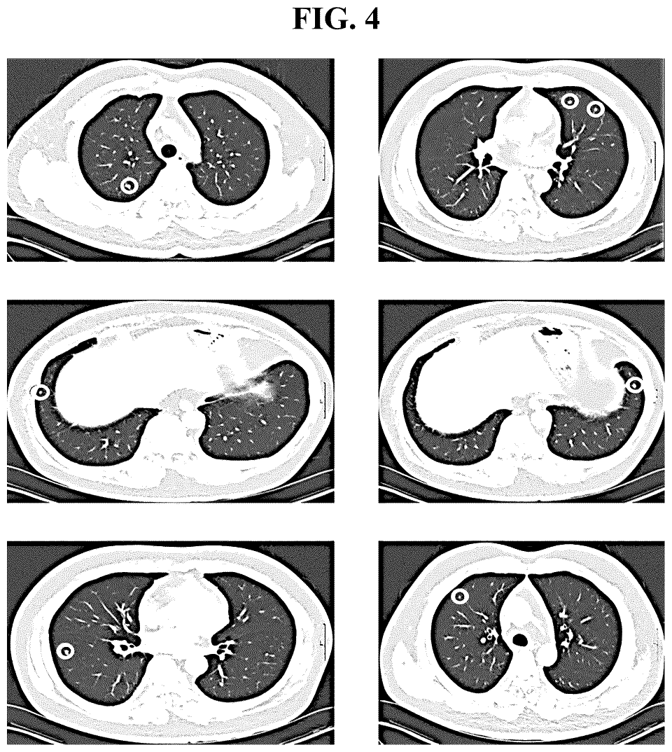

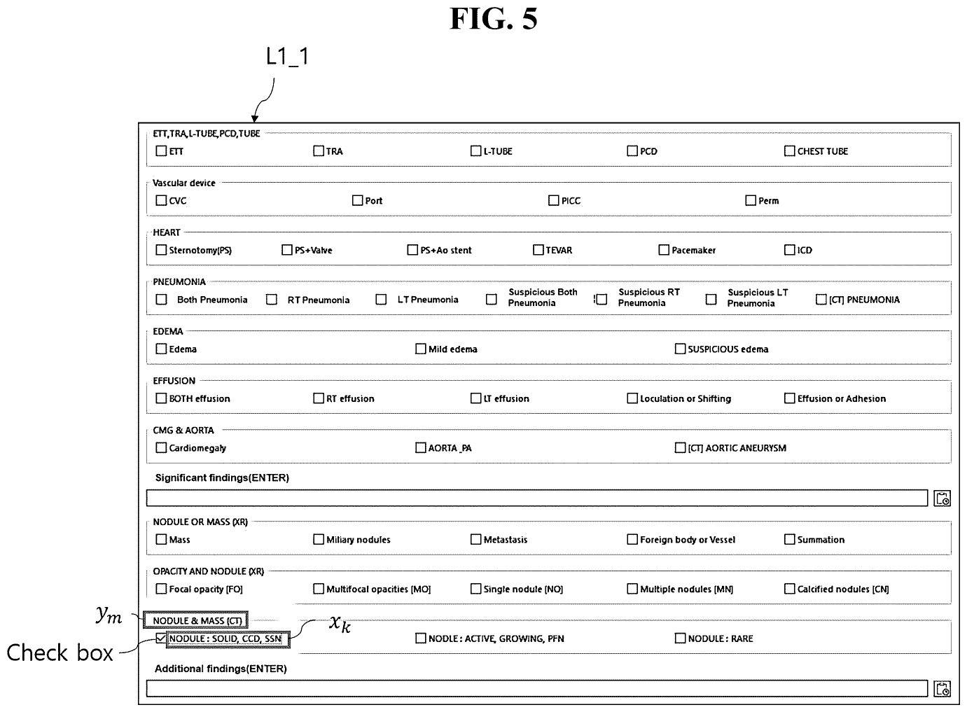

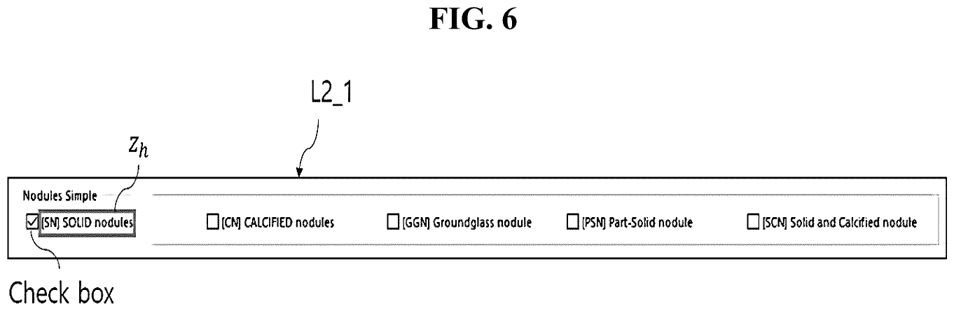

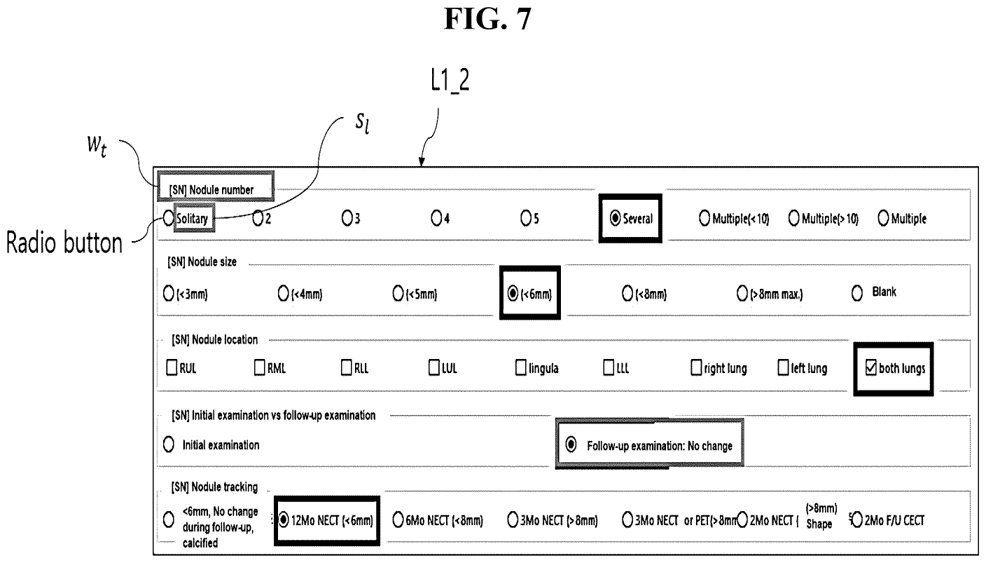

FIG. 4 is a diagram showing medical images for a chest examination according to an embodiment of the present disclosure. FIG. 5 is a diagram showing a primary layer of one set L1_1 according to an embodiment of the present disclosure. FIG. 6 is a diagram showing a secondary layer of one set L2_1 according to an embodiment of the present disclosure. FIG. 7 is a diagram showing a primary layer of a second set L1_2 according to an embodiment of the present disclosure.

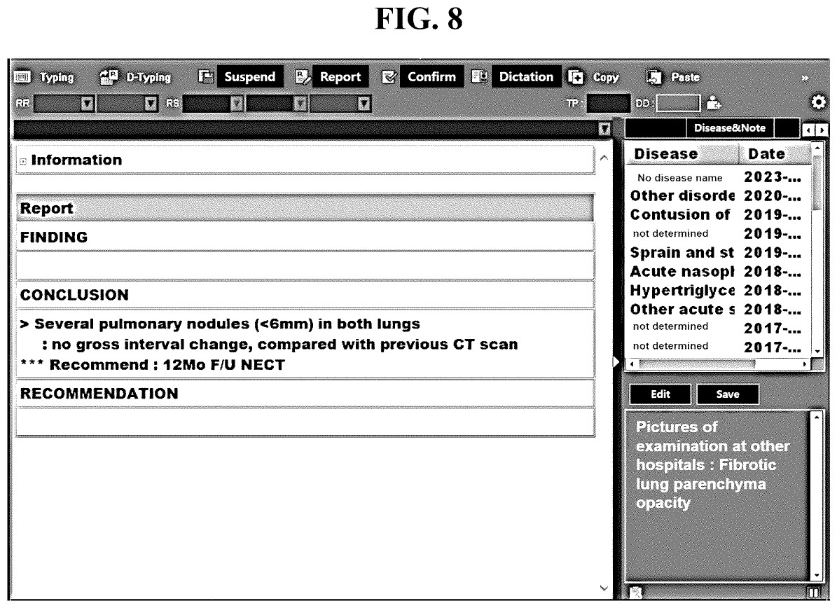

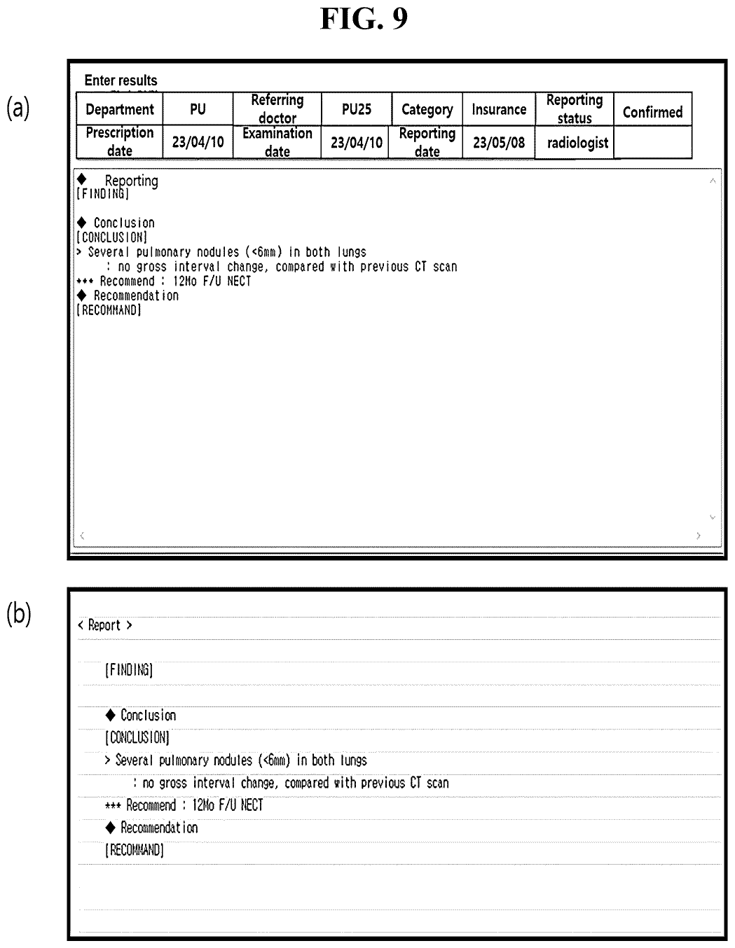

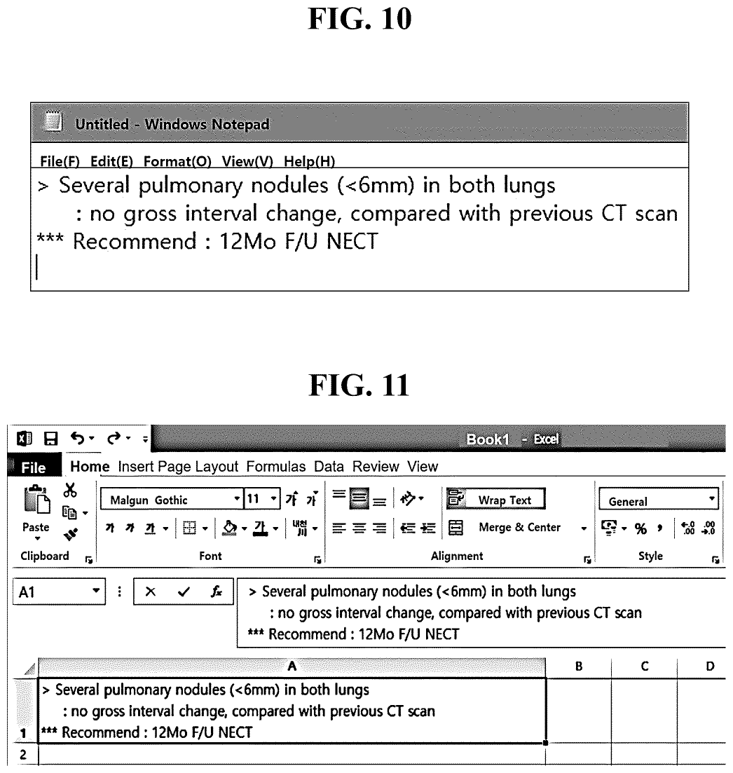

FIG. 8 is a diagram showing a picture archiving and communicating system (PACS) into which selection information is input according to an embodiment of the present disclosure. FIG. 9 is a diagram showing an electronic medical record (EMR) system into which selection information is input according to an embodiment of the present disclosure. FIG. 10 is a diagram showing a Windows notepad into which selection information is input according to an embodiment of the present disclosure. FIG. 11 is a diagram showing an Excel document into which selection information is input according to an embodiment of the present disclosure.

First, the present disclosure includes a recording medium 120 that can be read by a computer device 100 on which a program for performing a medical-image diagnostic report input method using a three-dimensional text data structure is recorded. For instance, the recording medium 120 may be a CD, a DVD, a hard disk, a Blu-ray disk, a USB, a memory card, a ROM, or the like. Furthermore, a method for inputting a medical-image diagnostic report using a three-dimensional text data structure of the present disclosure may be implemented by allowing at least one processor 110 in the computer device 100 to read the recording medium 120.

Referring to FIG. 1, a method for inputting a medical-image diagnostic report using a three-dimensional text data structure of the present disclosure includes a text information generation step S300 of generating, by at least one processor 110, text information T for medical-image reporting in a three-dimensional data structure, a user interface provision step S400 of providing, by the at least one processor 110, a user interface for the text information T, and a selection information input step S500 of inputting, by the at least one processor 110, only selection information selected from a user terminal 200 within the text information T provided to the user interface into a diagnostic report input field blank_R.

A medical image referred to in the present disclosure is an image that visualizes an inside of an examinee's body using various medical imaging devices such as X-rays, CT, MRI, and ultrasound. Furthermore, the text information T may be findings or a diagnosis itself when a radiologist reviews a medical image, and may be a medical term to express a disease area, a type of disease, a size, a location, a severity, a change during follow-up, and the like.

According to one embodiment of FIG. 3, first, the text information generation step S300 is characterized in that a text information input field blank_T is provided to input the text information T into the text information input field blank_T from the user terminal 200. A user, that is, a radiologist, may enter the text of the diagnostic report that is frequently used or essentially described based on his or her medical knowledge and experience into the text information input field Blank_T through the user terminal 200, thereby generating text information T in a user-customized manner.

Meanwhile, the user terminal 200 may connect to the Internet network to access a webpage related to radiology, including a Radiopaedia webpage, which is a Wikipedia-based radiology encyclopedia, and a thesis website. In the text information generation step S300, all or part of text may be copied from a specific part of the web page and pasted into the text information input field Blank_T by using a clipboard command, thereby facilitating an input operation.

However, for a radiologist with a lot of medical knowledge and experience, it may be useful to input the text information T in a customized manner from the text information generation step S300, but for a radiologist with little medical knowledge and experience, it may not be useful. That is, according to another embodiment of the present disclosure, the method may include a medical information acquisition step S100 of acquiring, by at least one processor 110, medical information based on web-scraping technology and a medical information grouping step S200 of grouping, by the at least one processor 110, the medical information according to preset criteria. Furthermore, the text information generation step S300 is characterized in that grouped medical information is provided from the grouping step S200 to allow a user to easily input the text information T into the text information input field blank_T.

First, in the medical information acquisition step S100, a uniform resource locator (URL) of a webpage related to radiology may be acquired to perform scraping on a specific part of a web page related to radiology as a target of web-scraping, thereby acquiring a plurality of items of medical information. Here, the webpage related to radiology may include a Radiopaedia webpage, which is a Wikipedia-based radiology encyclopedia or a thesis website. Furthermore, the medical information may be all or part of text from a specific part within a webpage related to radiology.

For instance, in case where ‘pelvic kidney with obstructing ureteric calculus’ and ‘normal right kidney in position and size’ are described in a specific part of the webpage related to radiology, the medical information acquisition step S100 may acquire medical information such as ‘pelvic kidney’, ‘ureteric calculus’, ‘normal’, and ‘right kidney’. Here, the medical information may include text indicating a disease area, text indicating a type of disease, text indicating a size, text indicating a location, text indicating a severity, text indicating a change during follow-up, and the like. In the medical information grouping step S200, the medical information may be classified into a disease area, a type of disease, a size, a severity, a change during follow-up, and other diseases that frequently occur along with the disease type may be classified and grouped.

Therefore, according to an embodiment of the present disclosure, the method may include the medical information acquisition step S100 and the medical information grouping step S200, thereby having a remarkable effect of allowing a doctor with little medical knowledge and experience to refer to the medical information as recommended keywords when entering the text information T for reading an arbitrary body part through the user terminal 200.

Next, it is characterized in that the text information generation step S300 includes an X-axis placement step S310 of placing an arbitrary item (ym) having a plurality of elements (x1, . . . , xn) on the X-axis, a Y-axis placement step S320 of placing a plurality of items (y1, . . . , yn) on the Y-axis, and a Z-axis placement step S330 of placing an auxiliary element (z) with respect to an arbitrary element (xk) in an arbitrary item (ym) on the Z axis. Here, n, m, and k are positive integers.

In one embodiment of FIG. 2, a three-dimensional data structure mentioned in the present disclosure is a structure including a primary layer L1_1 and a secondary layer L2_1. A layer referred to in the present disclosure may be a pop-up window provided in a user interface. Furthermore, most preferably, the text information generation step S300 is characterized in that text information T with a three-dimensional data structure is generated for each body part or for each imaging portion of a medical image.

Referring to (a) of FIG. 2, the primary layer L1_1 is arranged in two dimensions by including the X-axis and the Y-axis. Here, a plurality of elements (x1, . . . , xn) may be placed for each item (ym) on the X-axis, and a plurality of elements (y1, . . . , yn) may be placed on the Y-axis. Referring to (b) of FIG. 2, the secondary layer L2_1 may include the Z axis, thereby placing a plurality of auxiliary elements (z1, . . . , zn) with respect to an arbitrary element (xk) in an arbitrary item (ym). Therefore, in the text information generation step S300, text information T with a three-dimensional data structure may be implemented by using the primary layer L1_1 and the secondary layer L2_1.

Meanwhile, the three-dimensional data structure mentioned in the present disclosure may be a structure in which a plurality of sets are connected to one another, with the primary layer L1_1 and the secondary layer L2_1 as one set. As in the embodiment of FIG. 2, in a state where the primary layer L1_1 and the secondary layer L2_1 are generated as one set, another primary layer L1_2 may be connected to the previously generated secondary layer L2_1.

Here, in the X-axis placement step S310, an arbitrary first detailed element (wt) having a plurality of second detailed elements (s1, . . . , sn) for the arbitrary auxiliary element (zh) may be placed on the X-axis. Here, t is a positive integer. Furthermore, in the Y-axis placement step S320, a plurality of first detailed elements (w1, . . . , wn) may be placed on the Y-axis. That is, another primary layer L1_2 may be arranged with a first detailed element (w) and a second detailed element (s) in two dimensions. Although still another secondary layer L2_2 connected to another primary layer L1_2 is not shown in the embodiment of FIG. 2, the present disclosure may increase a number of sets including the primary layer and the secondary layer, thereby having an effect of increasing the scalability of text information T.

That is, the text information T of the present disclosure may be generated as a three-dimensional data structure including a primary layer L1_1 and a plurality of secondary layers L2_1, thereby providing a series structure in which the primary layer L1_1 and each of the secondary layer L2_1 in the set operate in a connected manner to be processed in order, and a parallel structure in which the plurality of secondary layers L2_1 operate in an independent manner to be processed simultaneously. Therefore, in the present disclosure, text information T may be generated in a three-dimensional data structure, thereby increasing scalability, and inducing a radiologist to more specifically write diagnostic findings.

In addition, in the text information generation step S300, a radio button or checkbox may be generated on one side of the text information T. According to one embodiment of the present disclosure, in the text information generation step S300, for a plurality of elements (x1, . . . , xn) with respect to an arbitrary item (y), radio buttons for single selection or checkboxes for multiple selection may be generated in a similar manner. Furthermore, for a plurality of auxiliary elements (z1, . . . , zn) with respect to an arbitrary element (x), radio buttons for single selection or checkboxes for multiple selection may be generated. Furthermore, for a plurality of second detailed elements (s) with respect to an arbitrary first detailed element (w), radio buttons for single selection or checkboxes for multiple selection may be generated in a similar manner. Here, single selection refers to selecting only one item from the user terminal 200, and multiple selection refers to selecting more than one item from the user terminal 200.

Next, in the user interface provision step S400, a user interface for the text information T is provided by the at least one processor 110.

Here, in the user interface providing step S400, the radio buttons or the checkboxes generated from the text information generating step S300 may be provided to allow the user to select the corresponding text information T through the user terminal 200.

In one embodiment of FIGS. 4 to 7, it is shown an embodiment of reading a chest examination. The medical image has been taken of an examinee's chest, including the lungs and heart. In a primary layer L1_1, the item (y), which is comprehensive content that can be read from the corresponding medical image, may include any one of the presence or absence of a specific device, a body part, and a type of disease such as vascular device, heart, pneumonia, edema, and nodule.

Furthermore, in the primary layer L1_1, an element (x) for each item (y) may be content that can be read in detail within the item (y), wherein the element (x) of the item nodule (y) may include any one of a disease severity, a disease location, and a mode, such as solid nodule (solid), newly formed nodule (active), and rare nodule (rare). If the item (y) is pneumonia, then the element (x) may include both pneumonia (both), right pneumonia (rt), left pneumonia (lt), both suspicious pneumonia (both suspicious), right pneumonia (rt suspicious), left suspicious pneumonia (lt suspicious), and the like.

Furthermore, in the secondary layer L2_1, the auxiliary element (z) may be content that can be read in more detail within the element (x), wherein the element of nodule (x) may include entire solid nodule (SN), calcified solid nodule (CN), ground-glass solid nodule (GGN), partially solid nodule (PSN), and solid and calcified nodule (SCN). Furthermore, in another set of a primary layer L1_2, a first detailed element (w) and a second detailed element (s) in the auxiliary element (z) is content that can be read in more detail within the auxiliary element (z). For instance, the first detailed element (w) may include nodule number, nodule size, nodule location, initial/re-examination, nodule tracking, and the like. Furthermore, the first detailed element (w) for a number of nodules may include second detailed elements (s) including 1, 2, 3, 4, 5, several, multiple below 10 or, multiple above 10, and the like.

Next, in the selection information input step S500, only selection information selected from the user terminal 200 within the text information T provided to the user interface by the at least one processor 110 is entered into the diagnostic report input field blank_R.

The user interface referred to in the present disclosure may be implemented on a graphic basis to facilitate visual confirmation and selection by the user in such a manner that the text information T and selection information are displayed on a display provided in the user terminal 200. Furthermore, the user terminal 200 refers to an electronic device installed with an operating system such as Windows OS, IOS, Linux, Android, or the like, and including a user input unit, a display, and a communication unit capable of Internet network communication. For instance, the user terminal 200 may be a personal PC, a tablet PC, a laptop, a smartphone, and the like.

Most preferably, in the selection information input step S500, text information T in which radio buttons or checkboxes provided on one side of the text information T are selected from the user terminal 200 among a plurality of text information T may be combined through a preset algorithm, thereby generating the selection information. According to one embodiment of the present disclosure, in the selection information input step S500, text information T in which a radio button or checkbox is selected becomes ‘1’, and text information T that is not selected becomes ‘0’, and only the text information T selected from the user terminal 200 may be listed in the order selected from the user terminal 200 or in the order arranged in each layer and generated as the selection information.

According to one embodiment of the present disclosure, the selection information input step S500 is characterized in that the selection information is inserted into the diagnostic report input field blank_R provided in another program using a clipboard command to improve input compatibility.

In the selection information input step S500, the selection information may be copied from a preset diagnostic report input field blank_R using the clipboard command and then pasted into a diagnostic report input field blank_R of the other program so as to be inserted into the diagnostic report input field blank_R. In the selection information input step S500, compatibility between other programs may be improved by using a clipboard command, and even if a number of characters in the selection information is quite large, it may be input into the diagnostic report input field blank_R at high speed.

According to another embodiment of the present disclosure, the selection information input step S500 is characterized in that the selection information is inserted into the diagnostic report input field blank_R provided in another program using a keyboard event simulation to improve input compatibility.

Most preferably, in the selection information input step S500, the keyboard event simulation may be implemented as if the user directly enters a diagnostic report input field blank_R provided in the other program using a Sendinput function. In the selection information input step S500, a keyboard event simulation may be used, thereby allowing the selection information to be easily input even in the other program where the clipboard command is restricted. Furthermore, the selection information including special characters may be easily input regardless of the arrangement and type of a keyboard used by the user.

In one embodiment of FIGS. 8 to 11, another program mentioned in the present disclosure may be a picture archiving and communicating system (PACS) generally used in a radiology department to read medical images, an electronic medical record (EMR) system used internally by a medical institution in connection with the picture archiving and communicating system (PACS), a Windows notepad provided by a Windows operating system, or a document program provided by various operating systems or a web browser. In other words, the present disclosure may improve compatibility with other programs used for conventional medical-image reporting to reduce a burden on a medical staff, including a radiologist, having to adapt to new programs and increasing the convenience of use.

Therefore, according to the present disclosure, when a radiologist with a lot of clinical experience writes diagnostic findings subsequent to medical-image review, he or she easily may easily write diagnostic findings by simply selecting text information T required for the diagnostic report without having to list medical terms one by one using a keyboard, thereby significantly increasing the efficiency of writing the diagnostic findings, as well as reducing a waiting time for a clinician and an examinee.

In addition, the present disclosure may be used as a writing guide when a radiologist with insufficient clinical experience writes diagnostic findings subsequent to medical-image review, thereby having a remarkable effect capable of writing diagnostic findings in the same manner as a radiologist with a lot of clinical experience regardless of his or her clinical experience.

Embodiments may be implemented by hardware, software, firmware, middleware, microcode, hardware description languages, or any combination thereof. When implemented in software, firmware, middleware, or microcode, the program code or code segments to perform necessary tasks may be stored in a computer-readable storage medium and executed by one or more processors.

Furthermore, aspects of the subject matter described herein may be described in the general context of computer-executable instructions, such as program modules or components that are executed by a computer. In general, program modules or components include routines, programs, objects, and data structures that perform specific tasks or implement specific data types. Aspects of the subject matter described herein may be practiced in distributed computing environments where tasks are performed by remote processing devices that are linked through a communications network. In a distributed computing environment, program modules may be located in both local and remote computer storage media including memory storage devices.

As described above, though the embodiments have been described with limited embodiments and drawings, those skilled in the art may make various modifications and variations from the above description. For example, although the above-described techniques are performed in a different order from that of the above-described method, and/or the above-described components, such as a system, a structure, a device, and circuit, are coupled or combined in a different form from that of the above-described method, or replaced or substituted with other components or equivalents, proper results may be achieved.

Therefore, other implementations, other embodiments, and equivalents to the claims are within the scope of the following claims.

1. A method for inputting a medical-image diagnostic report using a three-dimensional text data structure, the method comprising:
a text information generation step of generating, by at least one processor, text information for medical-image reporting in a three-dimensional data structure;
a user interface provision step of providing, by the at least one processor, a user interface for the text information; and
a selection information input step of inputting, by the at least one processor, only selection information selected from a user terminal within the text information provided to the user interface into a diagnostic report input field.

2. The method of claim 1, wherein the text information generation step comprises:
an X-axis placement step of placing an arbitrary item having a plurality of elements on the X-axis;
a Y-axis placement step of placing a plurality of items on the Y-axis; and
a Z-axis placement step of placing auxiliary elements with respect to an arbitrary element within an arbitrary item on the Z-axis.

3. The method of claim 1, wherein the selection information input step comprises inserting the selection information into a diagnostic report input field provided in another program using a clipboard command to improve input compatibility.

4. The method of claim 1, wherein the selection information input step comprises inserting the selection information into a diagnostic report input field provided in another program using a keyboard event simulation to improve input compatibility.

5. A computer-readable recording medium on which a program for performing the method of claim 1 is recorded.
